(19) United States
(12) Patent Application Publication
(10) Pub. No.: US 2017/0106082 A1
(43) Pub. Date: Apr. 20, 2017

(54) Personal Vaccine and Method of Making

(71) Applicant: Joseph Gerard Birmingham, Portland, OR (US)

(72) Inventor: Joseph Gerard Birmingham, Portland, OR (US)

(73) Assignee: Q21, Portland, OR (US)

(21) Appl. No.: 14/884,394

(22) Filed: Oct. 15, 2015

(51) Int. Cl. A61K 39/385

(52) U.S. Cl. CPC A61K 39/385; A61K 2039/6031

(57) ABSTRACT

A method for the creation of a personalized vaccine. Multiple and varied antigens in conjunction with heat shock proteins (and other protein chaperones) are generated by ionized gas lysing coupled with the separation, concentration, and purification of these chaperone protein-antigen complexes (CPAC) using insulator-dielectrophorsis (i-DEP)-based devices. The ionized gas uniquely forms more and varied chaperone proteins and chaperone protein-antigen complexes (CPAC) than prior art mechanical, chemical, electric or other lysing techniques. These CPAC generated by the ionized gas lysis and separated by i-DEP are electrospray-encapsulated by a biodegradeable polymer at the nano particle level to further enhance these personalized vaccines for accelerated immune system uptake. For the first time, sterile eradication of infectious pathogens and cancer (known or unknown to exist in the host) can be accomplished with multiple personalized vaccine treatments.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

The present invention relates to a personalized vaccine and its method of manufacture. Together, they introduce a breakthrough in technology for the treatment of infectious diseases, cancers, and autoimmunity reactions.

The human body is a remarkable, adaptive unit, capable of developing antibodies to seek out and destroy any type of pathogen or mutated cancerous cell therein—that it can find. Unfortunately, a host of evasion and anti-detection processes used by pathogens and cancerous cells allow for the growth and proliferation of that disease, infection or cancer and thus uncombatted by the host. A need exists for an evolving disease treatment. The most successful vaccines (e.g. polio, smallpox, measles) have been against causal pathogens that did not have sophisticated anti-immune defense mechanisms. Many pathogens including hepatitis C and human immunodeficiency (HIV) viruses, Mycobacterium tuberculosis, Helicobacter pylori, Plasmodium falciparum, have evolved complex immune system evasion strategies and require high-level effector T cell activation for their eradication. So far, these organisms have proved intractable to existing vaccination strategies. In addition, many diseases are not yet preventable by vaccination, and vaccines have not been fully exploited for target populations such as elderly and pregnant women. Vaccines are not yet available for hepatitis C virus (HCV), dengue, respiratory syncytial virus (RSV), cytomegalovirus (CMV), group B Streptococcus (GBS), Staphylococcus aureus, and Pseudomonas aeruginosa. Bioterrorism, emerging and re-emerging infectious diseases, changes in population demographics (e.g. senescence in the immune system of the elderly that are more exposed to nosocomially-acquired infections of antibiotic resistant bacteria), drive the need for new vaccine approaches.

If a pathogen can be isolated and transferred as an antigen to the lymph nodes, the innate/adaptive immune system stimulation of the dendritic cells can occur and an antibody will be produced that can eradicate the pathogen, essentially affecting a cure.

Henceforth, a preemptive strike vaccine that works as a personal, targeted immunotherapy would fulfill a long felt need in the medical industry. This new invention utilizes and combines known and new technologies in a unique and novel configuration to develop a personal vaccine to treat cancer, infectious disease and autoimmune reactions.

### SUMMARY OF THE INVENTION

The general purpose of the present invention, which will be described subsequently in greater detail, is to provide a personal vaccine treatment, (created while unaware of what specific infectious disease, cancer, or autoimmunity reaction exists within the patient) that is capable of provoking the body to create evolving antibodies to continually combat the evolving infectious disease, cancer or autoimmune reaction. To accomplish this end there are three novel devices each with their attendant methods of use for developing the personal vaccine treatment. These will be addressed herein.

The present invention has many of the advantages mentioned heretofore and many novel features that result in a new personal vaccine and its method of making which are not anticipated, rendered obvious, suggested, or even implied by any of the prior art, either alone or in any combination thereof.

In accordance with the invention, an object of the present invention is to create a preemptive, personal vaccine that targets all the existing infectious diseases, cancers, or autoimmunity reactions within the patient prior to the onset of any related symptoms and without the need for any medical diagnosis.

It is another object of this invention to provide a method of extracting a much higher number of, and more varied species population of chaperone proteins, chaperone protein complexes, chaperone protein antigen complexes or aggregates thereof that are extracted from a biological sample (lymph fluid, salvia, tissue (e.g. blood) or other bodily fluids) through the use of dielectrophoretic separation (i-DEP) in combination with ionized gas lysing.

It is a further object of this invention to provide multiple personalized treatments by a vaccine made by ionized gas lysings producing chaperone protein-antigen complexes (CPAC) vaccines that evolve as the pathogen or cancer attempts to find a resistance strategy so as to eradicate nosocomial infections and other diseases in a manner similar to smallpox.

It is still a further object of this invention to provide for an improved delivery system for chaperone protein-antigen complexes (mixed with a pharmaceutical grade excipient and coated with a biodegradable polymer at a nanometer-size diameter particle) to the body's immature dendritic cells to begin the immune system targeted response.

The above description will enable any person skilled in the art to make and use this invention. It also sets forth the best modes for carrying out this invention. There are numerous variations and modifications thereof that will also remain readily apparent to others skilled in the art, now that the general principles of the present invention have been disclosed.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

### BRIEF DESCRIPTION OF THE DRAWINGS

FIG. 1 is a front view of a first microfluidic card with a micropillar array embedded therein;

FIG. 2 is a front view of a second microfluidic card with a micropillar array embedded therein, offset from the first microfluidic card;

FIG. 3 is a front view of a microfluidic spacer card;

FIG. 4 is a side perspective view showing the alignment and order of the various three cards within a microstructured array;

FIG. 5 is a side perspective schematic view of a microstructured array used for non uniform field dielectrophoris;

FIG. 6 is a perspective side cut-away schematic view of a microstructured array used for non uniform field dielectrophoris;

FIG. 7 is a side perspective schematic view of a typical miniaturized ionized gas lysis device for the creation of chaperone protein-antigen complexes (CPAC);

FIG. 8 is a side perspective schematic view of the co-axial electrospray device;

FIG. 9 is an axial cross section view of the co-axial electrospray device;

FIG. 10 is a SEM photograph of Bg spores prior to ionized gas lysing; and

FIG. 11 is a SEM photograph of Bg spores after 15 seconds of ionized gas lysing treatment.

### DEFINITIONS

As used herein, the term “insulator-dielectrophoretic separation” (i-DEP) refers to a separation technique for biomaterials of various composition based on the motion of a polarizable particle in a suspending medium due to the presence of a non-uniform electric field where the voltage is applied using at least two electrodes that straddle an insulating structures array. When an electric field is applied across such an array, the presence of the structures creates regions of higher and lower field strength, i.e., dielectrophoretic traps. I-DEP systems do not lose their functionality despite fouling effects, which makes them more suitable for biological applications. I-DEP devices are used to perform insulator-electrophoresis, are inexpensive and can be fabricated from a wide variety of materials, including plastics.

As used herein, the term “reactive oxygen species (ROS)” refers to the chemical constituents of an ionized gas formed by intense electric field interaction with air. It includes but is not limited to species such as reactive nitrogen, reactive oxygen, hydroxyl radicals, peroxides and nitrogen oxides (including NO, NO₂, N₂O₅, and N₂O).

As used herein the term “ionized gas lysing” refers to the use of a high voltage discharge that produces an ionized gas also referred to a nonequilibrium plasma (here at a room temperature and atmospheric pressure) such that when a biological material is exposed to this ionized gas for a brief period, it breaches the membrane of a cell, opening the cell, breaking the cell membrane therein and allowing access to the fragmented cellular components. This is generally done for the purposes of generating antigens and chaperone proteins for subsequent collection and purification. Ionized gas exposure as performed and described herein serves to lyse the cells such that a larger, more varied population of molecules may be extracted.

As used herein the term “proteins” refers to a chain of amino acids regardless of the length and thus includes peptides.

As used herein the term “defensins” refers to a family of potent antibiotics made within the body by neutrophils (a type of white blood cell) and macrophages (cells that can engulf foreign particles). The defensins play important roles against invading microbes. They act against bacteria, fungi and viruses by binding to their membranes and increasing membrane permeability. Defensins are small peptides unusually rich in the amino acid cysteine that function as, host defense peptides. Defensins are also generated by the ionized gas exposure treatment disclosed herein.

As used herein the term “heat shock proteins” refers to a family of proteins found in virtually all living organisms, that are produced by cells in response to exposure to stressful conditions such as heat, UV light, mechanical stresses, and are produced during wound healing or tissue remodeling. Many members of this group perform chaperone functions by stabilizing new proteins to ensure correct folding or by helping to refold proteins that were damaged by the cell stress.

Heat-shock proteins are named according to their molecular weight wherein the Hsp60 family, would denote heat shock proteins on the order of 60,000 unified atomic mass units in size.

As used herein, the term “pathogen” refers to an infectious biological agent such as a virus, bacterium, prion, fungus, viroid, or parasite that causes disease/illness in its host. In its broadest terms it is anything that causes a disease. Some of the diseases that are caused by viral pathogens include smallpox, influenza, mumps, measles, chickenpox, ebola, and rubella.

As used herein, the term “antigen” refers to any structural molecule or linear molecule fragment that causes your immune system to produce antibodies against it. The antigen can be recognized by highly variable antigen receptors (B-cell receptors or T-cell receptors) of the adaptive immune system. Basically, an antigen serves as a target for the receptors of an antibody generated from an adaptive immune response. Each antibody is specifically selected for binding to a certain antigen because of random somatic diversification in the antibody complementarity determining regions. (Known as the “lock and key process” of connection between an epitope and a paratope.) Most tumors express antigens that could potentially elicit an immune response due to the expression of a mutant protein that gives rise to a novel epitope.

As used herein, the term “epitope” refers to the distinct surface features of an antigen. Antigenic molecules, normally “large” biological polymers, usually present surface features that can act as points of interaction for specific antibodies. Any such feature constitutes an epitope. Most antigens have the potential to be bound by multiple antibodies, each of which is specific to one of the antigen's epitopes. Using the “lock and key” metaphor, the antigen can be seen as a string of keys (epitopes) each of which matches a different lock (antibody).

As used herein the term “antibody” is a Y-shape protein produced by plasma cells. It is used by the immune system to identify and neutralize pathogens such as bacteria and viruses. The antibody recognizes the unique antigen molecule of the pathogen. Each tip of the “Y” of an antibody contains a paratope that is specific for one particular epitope (similarly analogous to a key) on an antigen, allowing these two structures to precisely, in proper alignment, bind together. Using this binding mechanism, an antibody can tag a microbe or an infected cell for attack by other parts of the immune system, or can neutralize its target directly.

As used herein, the term “heat shock” refers to a host of stressful conditions that a cell can be subjected to, including exposure to cold, starvation, hypoxia, water deprivation, heat, infection, inflammation, exercise, and exposure of the cell to toxins (ethanol, arsenic, trace metals, and UV light, etc.) Exposure to this causes a stress response which in turn causes the formation of heat shock proteins.

As used herein the term “chaperone protein” refers to proteins that would bind with other antigen proteins. Many chaperones, but by no means all, are heat shock proteins because the tendency to aggregate increases as proteins are denatured by stress.

As used herein, the term a “chaperone protein-antigen” refers to a chaperone protein that has bonded to an antigen to form a complex. Herein, this occurs when the chaperone protein is formed by ionized gas processes in the presence of a host of antigens, also formed in the ionized gas process.

As used herein, the term “chaperone protein complex (CPC)” refers to a molecular complex of chaperone proteins.

As used herein, the term “chaperone protein antigen complex (CPAC)” refers to a molecular complex of chaperone proteins and chaperone proteins bonded to another antigen protein. The antigen protein associated with the chaperone protein in a chaperone protein complex can be a naturally occurring protein, a protein produced by a genetically engineered cell, a protein produced by exposure of the cell to an ionized gas (or other lysing methods), or a synthetic protein. The unique characteristic of ionized gas exposure is that it will induce the production of heat shock proteins, other chaperone proteins and liberate of antigens and that it can even create recombinant protein complexes.

As used herein, the term “nanoparticles” or “nanometer-diameter sized particles or nano-sized” refers to a set or group of particles that have mean diameters in the range of 0.1 to 200 nanometers (0.0001 to 0.2 microns).

As used herein the term “microfluidic device” refers to devices (generally microfluidic chips) used in microfluidics in which a micro-channels network has been molded or patterned. Because of the various inlet and outlet ports, these microfluidic devices allow fluids to pass through different channels of different diameters, herein usually ranging from 0.5 to 500 μml/min. The micro-channels network is specifically designed for each application and the analyses desired. Microfluidic chips are the devices used in microfluidics in which a micro-channels network has been molded or patterned.

As used herein the term “pharmaceutical excipient” refers to an regulatory body (such as the USFDA) approved natural or synthetic substance formulated alongside, incorporated with, or applied to the active ingredient of a medication or a vaccine for the purpose of administering safe doses to the targeted recipient or for ease of administering the doses. These may be used as “bulking agents,” “fillers,” or “diluents” to add volume to concentrated medications or vaccines. They may also confer a therapeutic enhancement on the active ingredient in the final dosage form, such as facilitating drug absorption or solubility. They may aid in the handling of the active substance concerned such as by facilitating powder flowability or non-stick properties, or add in vitro stability such as prevention of denaturization over the expected shelf life. They include binders, antiadherants, coatings, colors, disintegrants, flavors, lubricants, glidants, preservatives, sorbents, sweeteners, vehicles and the like.

As used herein, the term “Taylor cone” refers to the phenomenon wherein when a small volume of liquid is exposed to an electric field such that the shape of the liquid starts to deform from the shape caused by surface tension alone. As the voltage is increased the effect of the electric field becomes more prominent. As the electric field approaches exerting a similar amount of force on the droplet as the surface tension does, a cone shape begins to form with convex sides converging to a pointed tip. When a certain threshold voltage has been reached the slightly pointed tip inverts and emits a jet of liquid. This is called a cone-jet and is the beginning of the electrospraying process in which ions may be transferred to the gas phase.

As used herein, “biological material” refers to blood, saliva, tissue, bodily fluids and bone marrow.

### DETAILED DESCRIPTION

Despite considerable research into therapies for infectious disease and cancer, these diseases remain difficult to diagnose and treat effectively. The present invention teaches a method and apparatus for developing a vaccine for treating cancer, infectious disease, and autoimmune reactions. More particularly, the present invention relates to a method and the various related apparatus used in recovering chaperone proteins or chaperone protein complexes from a limited sample. A mixture of different chaperone proteins complexed with proteins or peptides can be discretely recovered from a sample by a one-step method and apparatus employing insulator-Dielectrophoresis (i-DEP) coupled with ionized gas lysing and an optional further i-DEP processing to produce and isolate chaperone protein-antigenic complexes (CPAC). The focus of this patent is the extraction of these novel chaperone proteins—antigenic complexes (CPAC) generated by ionized gas lysis and the reduction of these to a nano-sized biodegradable polymer (PGLA) droplets that when reintroduced to the host, can cure infectious diseases and cancer. The overall vaccination preparation process is termed the enriched ionized gas homeostasis treatment (EIGHT) and is designed to allow the host's immune system to reestablish sterile eradication equilibrium after infection by pathogens, proliferation of cancer, and autoimmunity reactions in the body.

The present invention teaches three novel apparatuses and their attendant methods of use that may be combined or not to create a personal vaccine that enables the immune system to treat infectious diseases, cancer, and autoimmunity reactions. The entire process for the production of the personal vaccine is termed the enriched ionized gas homeostasis treatment (EIGHT).

The following brief description outlines the three apparatuses involved (the i-DEP device, the ionized gas lysing device and the nanoparticle electrospray encapsulation device), their individual methods of use as well as the overall process steps utilizing the abovementioned three devices, for the creation of a targeted immunotherapy vaccine developed from the vaccine recipient's own bodily fluids.

### Overview

The preparation and use of a customized, autologous vaccine against the tumors of individual patients is now feasible using tumor-derived CPAC complexes. For maximum effect the maximum number of antigen peptides must be extracted from a bodily fluid sample of the intended vaccine recipient (host). Prior art methods failed to generate a sufficiently diverse number of the chaperone proteins and chaperone protein complexes extracted from the host, that could be used as a complementary source of the chaperone protein-based vaccine. This invention teaches a method for collecting more and more varied chaperone proteins and chaperone protein complexes from a limited sample source. This allows for the creation of a much more efficient and successful vaccine.

Chaperone proteins and chaperone protein complexes may be obtained from any infected cell (including whole tissue, isolated cells and immortalized cell lines infected or transformed with an intracellular pathogen) whether infected with a virus, a bacteria, an intracellular bacteria, an intracellular protozoa, or any cancer cell including cancer that has metastasized to multiple sites, or cells circulating in the blood, lymph or other bodily fluids. (It is to be noted that bodily fluids incorporate biological tissues as well.)

### General Overview of the Personal Vaccine Production

Pathogens in the body (whether viruses, bacteria or microbes, including protein toxins), are captured by the body's white blood cells which send out chemical signals that cause the polarity of the white blood cells to change. Knowledge of the existing pathogens within the host's body (the intended recipient of the vaccine) and symptomatic indications of their presence need not exist at this time. The EIGHT process begins with the extraction of human biological material from a host, (generally blood, although biological material can also be obtained from tissue or other bodily fluid samples) followed by the enrichment of cells from the tissue or a bodily fluid (e.g. pathogens, tumor migrating cells, metastasized recirculating tumor cells or other cancer cells). This enrichment of cells is the separation and purification by insulator-dielectrophoresis (i-DEP) performed in an i-DEP device. (See FIGS. 1-6) For example, the changed polarity of the white blood cells (because they have engulfed the pathogens) provides the basis for the separation of these pathogen laden white blood cells and other biological material (such as tissue). The i-DEP device 2 uses an electric potential difference to segregate the white blood cells (or biological material) with their engulfed pathogens in them so that these are held suspended within the charged micropillar array 4 of the i-DEP device. For example, the blood is washed away with another fluid to further purify and separate the suspended cells. The voltage is then removed from the i-DEP device 2 and the cells collected are run through an ionized gas lysing device. This ionized gas lysing basically treats the cells and forms antigens and chaperone proteins. The antigens come from both the outside of the cells (and biological material) as well as from the outside surface of the pathogens. Chaperones from both the pathogens and the blood (or biological material) are formed by exposure to an ionized gas. The chaperones attempt to grab onto the peptide chains of the antigens to form a complex (CPAC). Thus, the ionized gas lysing produces exogenous and endogenous chaperone protein-antigen complexes (CPAC) from tissue-derived sources such as pathogen, pathogen-infected cells, and cancer cells. This ionized gas lysing enriches the cells in both the quantity and variety of CPAC present. This CPAC comprises a comprehensive collection of chaperone protein-antigen complexes including hsp 27, hsp28, (s-hsp), hsp40, hsp60, hsp70, hsp72, hsp 84/hsp86, hsp90, hsp100, hsp110, defensin, calreticulin, cathelicidins, BiP/grp78, grp75/mt, gp96, tumor suppressor P53, p21 CDK inhibitor, extracted foreign DNA, and proteins formed by exposure to peroxides, nitrogen oxides, and reactive oxygen species. This is generally followed by a second i-DEP process to retain, purify, and concentrate the biomaterials leaving mainly chaperone protein antigen complexes (CPAC). (This second i-DEP process is optional, but recommended, depending on the level of purification and concentration of the CPAC desired.) This second i-DEP generally involves a solution wash to eliminate (separate) unwanted useless agents of cellular material from the CPAC. Once the enhanced, purified CPAC is isolated, it is collected by removing the voltage to the i-DEP device. There is an optional step of mixing the collected CPAC with an approved, pharmaceutical grade excipient depending on the proposed method of reintroduction of the vaccine into the donor. (This may occur now or after the dry or wet particle CPAC encapsulations have been formed and collected.) These chaperone protein antigen complexes (with or without any excipient) are drawn into a first (inner) chamber of a coaxial syringe of the encapsulation electrospray device and a biodegrdable polymer is drawn into a second (outer) chamber of the same coaxial syringe. The coaxial syringe has a high voltage on the extractor electrode and an applied pressure to force out the fluids. The coaxial syringe is coupled to a source of pressure to form the encapsulation electrospray device. Therein, when a small volume of electrically conductive liquid chaperone protein antigen complexes are exposed to an electric field and forced to the exiting end of the syringe, a cone shape begins to form and when a certain threshold voltage has been reached the slightly pointed tip of the cone inverts and emits a cone-jet of liquid. This is beginning of the electrospraying process and the CPAC laden droplets are transferred to the gas phase. The Taylor cone phenomenon allows the breaking off of the droplets at a prescribed size because of the applied voltage differential. The CPAC droplets fall onto a plate as dry biodegradable polymer encapsulations (with the encapsulation layer approximately 0.5 nanometers thick) having a mean diameter in the nanoparticle range. The CPAC or CPAC/excipient mixture is now nanoparticle-sized which have their exterior surfaces coated by the biodegradable polymer (e.g. PLGA). This coating enhances the targeting of the CPAC or CPAC/exicipient mixture for reception by the lymph system. At this point the vaccine can be reintroduced into the body (generally by injection) as a personal vaccine for disease treatment. (Optionally, at this time the previously discussed excipient may be added.) Upon reintroduction into the donor, the donor's white blood cells see the vaccine as foreign bodies and transport them to the lymph nodes where the immature dendritic cells engulf the CPAC and develop the appropriate immunization defenses such as developing antibodies for the specific pathogens that were in the host's original biological material. It is to be noted though, that the i-DEP extraction process in certain situations may be eliminated and the biological material, upon extraction, may be subjected to ionizing gas lysing producing the CPAC's that can then be reintroduced to the host. Similarly the i-DEP process may be used prior to the ionized gas lysing, after the ionized gas lysing or both and the diodegradable polymer electrospray may or may not be utilized with any combination of the above processes.

### Basic Steps in the Enriched Ionized Gas Homeostasis Treatment (EIGHT)

The novel process of enriched ionized gas homeostasis treatment (EIGHT) to develop an immunotherapy vaccine of chaperone protein-antigen complexes that are used, upon reintroduction into a donor's body, to generate T cells and other cells reactive to a chaperone protein antigenic complex molecules, utilizes all or most of the following abbreviated steps (which will be discussed in detail hereafter):
1. Extracting a sample of bodily fluid or tissue containing disease causing biomaterials from an intended vaccine recipient;
2. Passing said biological material through an insulator-dielectrophorectic device's microstructured array to separate and concentrate biological materials including cancer cells and pathogens;
3. Purifying and separating the disease causing biological materials by flushing away the remaining sample and retaining the remaining purified biological material that is suspended between the charged micropillars of the i-DEP microfluidic device's microstructure array, then removing the electric charge from the i-DEP device and retrieving the remaining purified biological material (in flushing fluid).
4. In vitro, ionized-gas lysing of said concentrated biological materials in the flushing fluid to produce a chaperone protein antigenic complex (CPAC) by the noncovalent interaction of an antigen molecule and a chaperone protein;
5. Extracting said chaperone protein-antigenic complexes in the flushing fluid,
6. Passing said extracted chaperone protein antigen complexes through said insulator dielectrophorectic device's microstructured array a second time, to separate and concentrate said extracted chaparone protein antigen complexes (CPAC);
7. Extracting said concentrated chaperone protein antigen complexes by removing the electric charge from i-DEP device and retrieving CPAC solution (in flushing fluid).
8. Drawing the CPAC solution up into one chamber of the electrospray coaxial syringe and drawing up a biodegradable polymer into the second chamber of the electrospray encapsulation device's coaxial syringe.
9. Applying a high voltage to the coaxial syringe (or extractor electrode) and applying pressure and sufficient electric fields to form nanosized droplets from the CPAC solution coated with the biodegradable polymer to form nanoparticle-sized dry encapsulations of the CPAC.
10. Collecting said coated, concentrated chaperone protein-antigen complexes, for the grounded vessel.
11. Reintroduction into the host, generally by injection after drawing the nanoparticle encapsulated CPAC into a syringe.
12. Optional possible inclusion of an excipient at various stages of the process.

The following disclosure focuses on the three apparatuses and their attendant methods of use during the various intermediary stages of the creation of the personal vaccine, prior to its reintroduction into the host.

Steps 1, 2 & 3—Obtaining the Disease Causing Biological Materials with i-DEP

Pathogens (infectious agents) enter the body (whether viruses, bacteria or microbes, including protein toxins) and are captured by the body's white blood cells which see these pathogens as foreign bodies. The white blood cells send out chemical signals that cause the polarity of the white blood cells to change. Some pathogens are not recognized by the white blood cells, however they do carry an electrical charge thereon. This polarity provides bases for the separation of the pathogen laden white blood cells, circulating tumor cells, and pathogens. The separation of white blood cells that have encapsulated pathogens (such as a cancer cells) pathogens and migrating cancer cells therein, are of particular value in medicine.

Existing cell sorting approaches, such as Fluorescence Activated Cell Sorting (FACS), magnetic activated cells sorting, and chemically functionalized pillar-based microchips, have shown promise as techniques that isolate rare cells, but are based on known receptors expressed on the surface of the membrane. As opposed to other techniques that rely on information at the membrane surface, i-DEP can noninvasively sort populations through differences within the interior of cells, as well as their exterior.

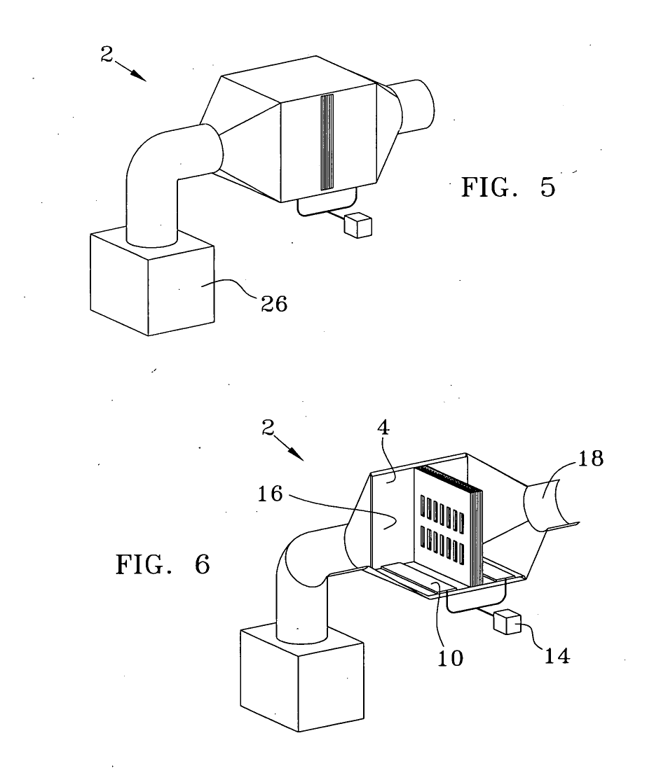

With i-DEP, separation can occur based on the different electric charge distribution on or within the cell (polarity). The dielectric properties of cells depend on their type and physiological status. For example, MDA-231 human breast cancer cells were found to have a mean plasma membrane specific capacitance of 26 mF/m², more than double the value (11 mF/m²) observed for resting T-lymphocytes. When an inhomogeneous AC electric field is applied to a particle, a dielectrophoretic (DEP) force arises that depends on the particle dielectric properties. Therefore, cells having different dielectric characteristics will experience differential DEP forces when subjected to such a field and the use of differential DEP forces allows for the separation of several different cancerous cell types from blood in an i-DEP device. These i-DEP devices 2 (FIGS. 5 and 6) are thin fluid chambers 4 of alternating micropillars 6 and cutouts 8 (voids) (FIG. 1) to create a non-uniform electric field for dielectrophoretic separation. DEP forces generated by the application of AC fields to electrodes 10 embedded at the entrance and exits of the electrically-separating microstructure array (microfluidic card) 12 (FIG. 4) are used to influence the rate of elution of cells from the chamber 4 by electrohydrodynamic forces within a parabolic fluid flow profile. The micropillars 6 are arranged in rows and are spaced apart from each other to define fluid passageways (FIG. 4).

Unlike other micropillar configurations, these passageways are much larger than the cell diameters (around 10⁻⁶ m) such that the separation is due to electrical means. When a bioparticle laden fluid stream flows through the micropillars, particles electrically migrate by interaction with nonuniform electric fields, and become retained or trapped in a particular area of flowing fluid. The fluid stream is deflected aside and flows around the micropillars 6 enabling the biomaterials to see many electric field strengths. The nonuniform AC electric fields cause the deposited biomaterials to migrate to specific micropillars.

In order for the fluid containing a protein sample to be concentrated it will flow through the electrified microstructured array 12. To effect separation, the electrical power will be applied to a micropillar array 12 as a nonuniform electric field from a electrical power supply 14 (FIG. 6). This i-DEP device enables separation of viruses, proteins, and other biomaterials. The retention of the immobilized protein complexes and CPAC at a certain location downstream of the initial micropillars is predictable.

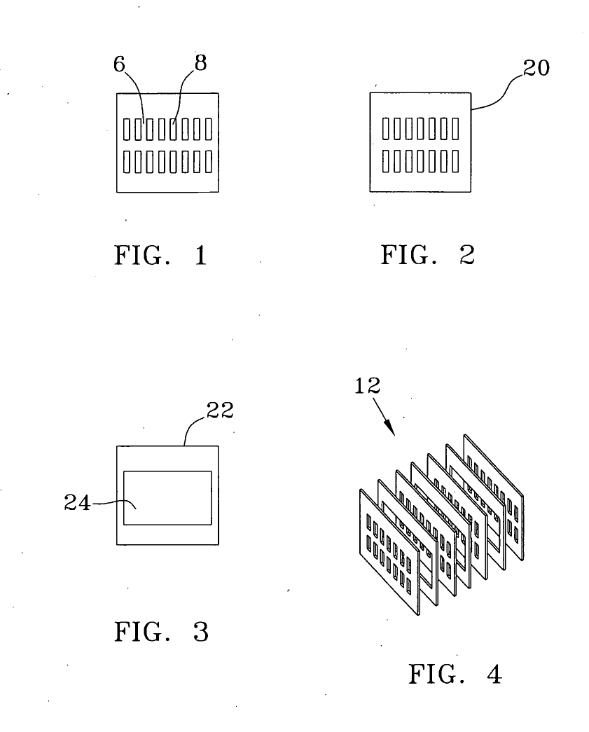

The general arrangement of the i-DEP microfluidic device 2 is best illustrated in FIGS. 1-6. FIG. 6 is a schematic view of an i-DEP microfluidic device 2. An i-DEP microfluidic device 2 (with sheet architecture) has a microstructured, micropillar array arranged thereon. There is a dielectric substrate base of the microfluidic device 2, with an electric charge present at the influent and effluent of the microfluidic device 2 via a set of imbedded electrodes 10 (generally planar) connected to an AC field generating power source 14. The electrodes 10 are coated with an inert material to prevent contamination of the biological material. (In the preferred embodiment the coating is Teflon present in a thickness of approximately 150 nm, and there is only two electrodes used, although in alternate embodiments there may be additional electrodes placed between the influent end 16 and effluent end 18 of the microstructure array 12.) In the preferred embodiment, a microstructured array 12 is based on a series of at least three microstructure sheets. These sheets are of two types, a micropillar sheet 20 (FIGS. 1 and 2) having a series of spaced slits therethrough 8 so as to form voids and pillars 6, and a spacer sheet 22 with at least one large central orifice 24. In the preferred embodiment, these microstructure sheets 20 and 22 are arranged in an alternating fashion such that there is a spacer sheet 22 between adjacent micropillar sheets 20 and the voids and pillars formed in adjacent micropillar sheets 20 are offset so as to cause the fluid (laden with biological material) and driven through the i-DEP microfluidic device 2 by a fluidic pump 26, to flow around the pillars 6 through the slits 8 from the influent to the effluent ends of the device 2. (However, it is known in the art that alternate structural embodiments may be utilized and would function provided that there was a sufficient area for the passage of fluid between pillars 6 and the driving electric field was changed to compensate for the configuration. In the most simple physical form there would be no spacer sheets 22 and but one micropillar sheet 20. In the preferred embodiment spacer sheets 22 were utilized because for economic purposes and for ease of fabrication, all micropillar sheets 22 were identical and their void necessitated a spacer sheet 22 to enable a flow path between adjacent microsheets. Micropillar sheets 20 with offset slits 8 may also require spacer sheets 22 depending on the specifics of the slit size, location etc.)

The concentration, segregation, and retention of even proteins with slight differences (e.g. prions) is based on a dielectrophoretically-enhanced microstructured array using nonuniform alternating current (AC) fields. The concentration of biomaterials within the microstructured array is designed for separating and purifying protein and their complexes (e.g. defensins, CPAC, prions, antibodies, and antigens). Concentration is accomplished by extraction of these from a solution of bodily fluid (generally blood). The protein complexes remain bound to this micropillar region until power is terminated. The separation of protein complexes can occur when proteins change their conformation, their dipole moment and polarizability, change slightly, and allow separation due to their migration differences within a nonuniform electric field imposed on the microstructured array.

The retention of biomaterials onto the microarrays was accomplished with nonuniform alternating electric fields applied to a microstructured array. Physical characteristics of cells such as polarizability, dipole moment, and resonance frequency facilitate separation when an electric field is applied to bioparticles. For example, cells along with their lysates (and their membranes and subcellular components) have dipole moments that are due to the separation of existing or induced particle charges. The charge separation of a particle causes the bioparticle to first align itself with the field. Then, the aligned particle migrates if the applied electric field has the appropriate conditions. The application of the nonuniform alternating electric field within the conductor-bounded micropillar array has the additional benefit of extending the electrode's influence within the captured biomaterials and preventing detours of the electrodes ultimately facilitating the separation of bioparticles.

Biomaterial behavior in a nonuniform alternating electric field includes:
Progressive motion (dielectrophoresis) and deposition of bioparticles at the electrodes
Orientation of bioparticles along and across force lines,
Formation of bioparticle cooperative chains,
Rotation of separate bioparticles,
Cooperative rotation of bioparticles relative to each other,
Electrotransformation of bioparticles.

In general, the bioparticle interaction with an electric field is dependent on the quantity and motion of their electric charges. The external nonuniform alternating electric field excites an oscillation of electric charges that causes ohmic losses, dielectric losses, molecule dipole relaxation, and similar effects. It is known that the oscillation amplitude peaks occur at resonant frequencies. The intrinsic bioparticle charge can be envisioned as a multipole. In accordance with the number of charges (Qₒ) and their spatial arrangement relative to each other can be described for multipoles in equation 1

Q₀=do+d1+d2+ . . . dn (1)

where the first multipole is a dipole, the second is a quadrupole, and subsequent higher order dipoles. The bioparticles such as protein complexes, cells, viruses, and bacteria are known to have a polarizability and dipole moment. The behavior in the nonuniform alternating electric field of a bioparticle affects its dipole moment and the induced processes internal to the bioparticle. Similarly, conventional electrophoresis accomplishes the separation of bioparticles as a function of the magnitude of their static electric field; However, if bioparticles are neutral, their separation in the electrostatic field is not feasible. Equation 2 illustrates another source of polarizability in a bioparticle is the dipole (d). The dipole system involves two or more charges opposite in sign. The quantitative characteristic is a vector of dipole moment (d) directed from the negative charge to the positive charge:

d=q1 (2)

where q is the absolute value of the electric charge and 1 is the vector describing the direction of the charge (from the negative to the positive). The bioparticle dipole moment may be constant or induced. The induced dipole moment is caused by the redistribution of electric charges (electrons, ions, etc.) over the bioparticle volume under the influence of an external electric field. The time required to return to the conditions before the application of an electric field condition is described as the relaxation time. The value of the induced dipole moment is proportional to the electric field strength and the bioparticle polarizability found in equation 3:

d=∈₀α(ω)E (3)

where ∈ₒ is the dielectric constant, α(ω) is the bioparticle polarizability coefficient, and E is the value of the electric field strength. The total dipole moment (d₀) of the bioparticle is defined by equation 4:

d₀=dc+dθ+di+dor (4)

where dc is the constant dipole moment, de is the dipole moment defined by electric polarization (with a relaxation time is τ˜10⁻⁽¹⁰⁻¹⁵⁾ s), di is the dipole moment defined by ionic polarization (with a relaxation time is τ˜10⁻⁽³⁻⁸⁾ s), dor is the dipole moment defined by orientation polarization (with a relaxation time is a variable from a fraction of second to several minutes τ˜10⁽⁻¹⁻²⁾ s). This complicated polarization coefficient with the different relaxation times is a function of the electric field frequency.

The bioparticle in the external electric field is polarized and the dipole moment dₚ is proportional to the polarizability coefficient of the particle αₚ(ω) and the value of the electric field strength E is induced in this particle as found in equation 5:

d_{P}=αₚ(ω)∈₀Eₓ−α_{w}(ω)n∈₀Eₓ (5)

where αₚ(ω) is the bioparticle polarizability, α_{w}(ω) is the polarizability of water molecule, n is the number of liquid molecules in the water volume displaced by the bioparticle, Eₓ is the electric field strength.

The nonuniform alternating electric field produces a force (Fₑₗ) is exerted on the particle with the dipole moment that tends to move the particle as found in equation 6:

$${F}_{el} = ( {α}_{p} ( ω ) - {α}_{w} ( ω ) n ) {ɛ}_{0} {E}_{x} \frac{d {E}_{x}}{d x} \qquad ( 6 )$$

where dEₓ/dx is the gradient of the nonuniform electric field strength [grad E]. The drag force (Stokes force Fₛₜ) value is defined by the viscosity of the solution, bioparticle size, and velocity of the fluid flow, is counteracted by the force, Fₑₗ as shown in equation 7:

Fₛₜ=6πη_{w}Vr (7)

where η_{w} is the viscosity of water, v is the velocity of particle motion, and r is the bioparticle radius. Equating the viscous force (Fₛₜ˜1 10⁻¹²N) with the nonuniform alternating electric field force (Fₑₗ) we have in equation 8:

$$( {α}_{p} - {α}_{w} n ) {ɛ}_{0} {E}_{x} \frac{d {E}_{x}}{d x} = 6 πηυ r \qquad ( 8 )$$

The results indicate that the difference between the bioparticle polarizability (αₚ) and the solution medium (α_{w}) produces a force that may be positive (directed to the micropillar electrode) or negative (directed from the micropillar electrode).

The polarizability of the medium and the bioparticle depends on their individual properties such as size, shape, temperature, chemical composition, surface conductance, dielectric constant, quantity of water, and quantity of charged and dipole molecules. This suggests that we may choose a frequency of the electric field such that Fₑₗ for the bioparticles is positive or negative. The motion of bioparticles in a nonuniform alternating electric field depends on the frequency. For example, at a low frequency (5-15 kHz), the bioparticles move to the region of minimum electric field strength. It has been found experimentally that at low frequency with electric fields at U=10 V, water electrolysis is found. At intermediate frequencies, the bioparticles are motionless or rotate around their own axis. At high frequencies (25 kHz-1 MHz), the bioparticles move to their singular micropillar electrode and are retained in a zone. The novel basis of this invention is that when proteins change their conformation, their dipole moment and polarizability change slightly and allow separation due to their migration differences within a nonuniform electric field imposed on the microstructured array. These changes in the bioparticle characteristics exhibit changed polarization and motion in the electric field.

It can be seen from equation that solving equation 8 for the polarizability coefficient of the particle αₚ(ω) can be easily rearranged into equation 9:

$${α}_{p} = \frac{6 πη vr - {α}_{w} n {ɛ}_{0} EgradE}{{ɛ}_{0} EgradE} \qquad ( 9 )$$

The values of the dipole moment (6 10⁻²⁴ C m) and the polarizability coefficient (4.2 10⁻¹⁸ m³) can be determined when one knows the biopartcile position, electric field strength E (6.7 10⁴ V/m), gradient of the electric field (1.2 10⁹ V/m²), velocity [v] (1 10⁻⁶ μm/s) of the cell motion, cell radius a (8 10⁻⁶ m) with a cell volume (2.1 10⁻¹⁵ m³). The dielectric constant of vacuum is defined to be (∈₀=8.85 10⁻¹² F/m). The cell under the action of the electric field force moves in the viscous fluid (water) with a viscosity of (η_{w}˜10⁻³Πa.s.) and a coefficient polarizability of water molecule α_{w} of (3.37 10⁻³⁷). The number of water molecules n in the cell volume (for example 9.9 10¹² for human erythrocytes) is defined by calculation in equation 10:

$${n}_{p} = \frac{4 π r}{3 * 27 * {10}^{- 30}} \qquad ( 10 )$$

where θ is the volume of the water molecule (and is equal to 27 10⁻³⁰ m³). The velocity of bioparticle cell motion may be defined by registering its traversed distance in a fixed time. The strength and gradient of the electric field are calculated by numerical solutions of the Laplace equation 11:

∇²U=0 (11)

The equation is solved under the boundary conditions determined the geometric parameters of the electrodes (height, width, radius, and distance between electrodes (e.g. 1 10⁻⁴ m) and voltage at the electrodes.

Various bioparticles can be concentrated by varying the voltage and frequency on each electrode. Each kind of bioparticle is concentrated in its own row of micropillars on a microstructured array and removed by the action of the flow drag force. The extension of the influence of the electrodes into the fluid will be accomplished with the conductor-coated micropillars.

Insulator-based dielectrophoresis (i-DEP) is a technique where insulating structures function as “obstacles” when applying an electric field, and their presence bends the electric field creating regions of higher and lower field intensity (i.e. a non-uniform electric field). Specifically, arrays of insulating structures were used to trap specific cells from bodily fluids.

The use of AC fields with insulating micropillars with constrictions that are a few microns wide concentrate and i-DEP trap bioparticles of interest. At low frequencies, some DNA particles can be pulled out of DEP traps (or constrictions) by electrophoretic forces. At higher frequencies, the i-DEP force was greater than the electrophoretic force, which allows for bioparticles of interest to become dielectrophoretically immobilized at the constrictions. Larger bioparticles trap sooner than smaller bioparticles. A high-throughput operation requires a flow-through system. The bioparticles, (i.e. DNA, proteins, blood components, etc.) are not denatured by i-DEP trapping or agglomeration at a constriction. Concentration factors for bioparticles from 8 to 240 times the feed concentration were obtained with a processing time of around 2-15 minutes, making this technique a fast tool for sample concentration. A batch flow system where bioparticles suspended in a medium or bodily fluid as a plug flow, flow through the device, it leaves the segregated biomaterials at different locations. These materials can be eluted as a plug of concentrated biomaterial for further analysis and treatment by terminating power. Epifluorescence microscopy was used to verify that the release of the cell contents into the microchannel by observing the fluorescent pEGFP-β-actin construct, and demonstrated that the remaining cell debris can be retained between the electrodes by i-DEP after the cell lysate contents have been released.

Bioparticles such as proteins, cells, viruses, and other biomaterials can clearly be segregated on a microstructured array. The segregated immobilized target protein is transferred to a specific zone by imposing an electric field; thereby producing a variation of the Western blot using conventional separation methods. The optimum nonuniform electric fields have enhanced the purification, retention, and concentration of the desired bioparticles.

Result show that the trapping of proteins is independent of the scale with respect to the geometry of an i-DEP device as long as the applied electric field remains constant. Voltage dependency on concentration distributions has also been explored in both micro-scale and nano-scale device geometries. To achieve i-DEP trapping of the proteins, nano-scale geometry is a better selection, as the voltage necessary to generate the required electric field (2.5 MV/cm) is 10⁵× lower compared with the voltage required to generate the same field in the micro-scale device. Additionally, low pH and high conductivity optimize the separation in an i-DEP device.

In the preferred embodiment i-DEP, teflon is used for the microstructrued array, the micropillars 6 are arranged 150 microns center-to-center, the micropillars are 100 microns in diameter, and 100 microns high to form a microfluidic device. The entire microstructured array (pillars and voids) is approximately one square centimeter. A 200 V high voltage sequencer was used to apply AC fields (10 Hz-10 MHz) to gain additional i-DEP control through frequency modulation of the Clausius-Mossoti factor. A flow of approximately 1 to 10 ml/min approximately, and a normal sample gathering time of around 2-15 minutes is used. (This technique is a fast tool for sample concentration.) Concentration factors for bioparticles from 8 to 240 can be obtained. (Concentration factor=ml of material processed X the percentage recovery rate.) The actual flow and applied AC voltage is optimized for the specific biomaterial sought by adjustments indicated by microscopic visual indications taken through a transparent top plate of the microfluidic device. Upon termination of the applied AC voltage, the biomaterials can be eluted as a plug of concentrated biomaterial for further analysis and treatment.

One of the novel features of this disease treatment approach is that specific pathogen containing cells can be extracted from a bodily fluid. For example, cancer cells or circulating tumor cells (CTCs) can be found in the blood of cancer patients. CTCs are 10⁶ rarer than white blood cells, making their capture and concentration particularly challenging. Over 10% of CTC do not have a tumor of origin found. In addition, pathogens circulate in the blood stream. The i-DEP is used to concentrate these coveted lysate targets. Personalized vaccines can target and eradicate diseases before they are diagnosed. The eradication of multiple diseases can be envisioned.

Generating cells in sufficient numbers and with varied pathogens cells is challenging, mostly due to their low number compared to background cells. For example, in screening for Circulating Tumor Cells (CTCs) to detect cancer, there are only a few CTCs per mL of blood, which includes approximately a billion red blood cells and a million white blood cells. Specifically, it has been reported that there are less than 5 CTCs per 7.5 ml blood. With i-DEP's ability to separate out cells based on both internal and external charges on the blood cell it provides an efficient methodology for separation. In operation, the rare cells population is trapped due to positive DEP force, while the background cells pass through the microdevice without trapping. With i-DEP large quantities of sample can be separated or enriched rapidly. Hence, milligram or more quantities of enriched chaperone proteins and complexes can be obtained from a gram of starting material embedded in a large amount of bodily fluid such as blood.

I-DEP enriches samples containing chaperone protein complexes and i-DEP fractions enriched for chaperone proteins contain chaperone proteins that exist in multimeric forms.

### Steps 4 and 5—Lysing the Antigens for Maximum Concentration and Variation

A protein, protein complexes, or CPAC must be concentrated and slightly purified to remove unproductive immunogens (e.g. cellular components that only elicit an immunologically response without any therapeutic benefit). Cell lysis is a process by which the cell membrane is breeched so that the intercellular substances such as proteins, nucleic acids, and other components can be fragmented and extracted for examination or subsequent use. Here, ionized gas lysate proteins along with antigens can be complexed (noncovalently-bonded) with a chaperone protein to produce a vaccine specific for the disease condition. Cell lysis must be rapid to prevent further biochemical changes, selective to avoid denaturing biomaterial of interest, and here, specific, to induce the concentrations of chaperone proteins-antigen complexes needed to produce individualized vaccines.

There are several ways to accomplish this including chemical lysis, mechanical lysis, electrical lysis, laser lysis, thermal lysis and sonication (ultrasonic lysis). However, the amount and variation of heat shock proteins, defensins and other chaperone protein-antigen complexes that can be retrieved per unit sample by the aforementioned lysing methods is far below that which can be recovered by atmospheric pressure nonthermal ionized gas lysis. Rapid lysis of human cells/tissue by ionized gases provides heat shock proteins, defensins, and other chaperone protein-antigen complexes.

Ionized gas lysing combines several lysis techniques heretofore accomplished by the individual methods discussed below, into a single process that mimics the results all of these methods and in doing so, generates more and varied chaperone protein-antigen complexes (CPAC) than any other individual lysis technique. The ionized gas lysing device uses electric fields to generate more and varied CPAC by the combination of the following multiple lysing techniques:

Chemical Lyse: Production of reactive oxygen species (ROS) that chemically lyse and initiate multiple cell processes,

Thermal Lyse: Ohmic heating to elevate the temperature of the cells,

Electrical Lyse: Electroporation to force some proteins into the extracellular environment,

Mechanical Lyse: “Microspears” are embodied by ionized gas microstreamers that create holes in the cells.

For more than one hundred years scientists have reported that direct and alternating electrical currents can kill or inhibit the growth of bacteria and yeast. Repetitive high voltage pulses have been recommended for continuous sterilization of liquid streams, however, researchers report that cells killed by pulsed electrical fields are not disintegrated, and bacterial spores, molds, and viruses are relatively insensitive to high voltage pulsing. The miniaturized discharge initiates an ionized gas using a solid-state transformer about the size of a deck of cards. Biological aerosols are lysed through the interaction of the ionized gas products with the cells.

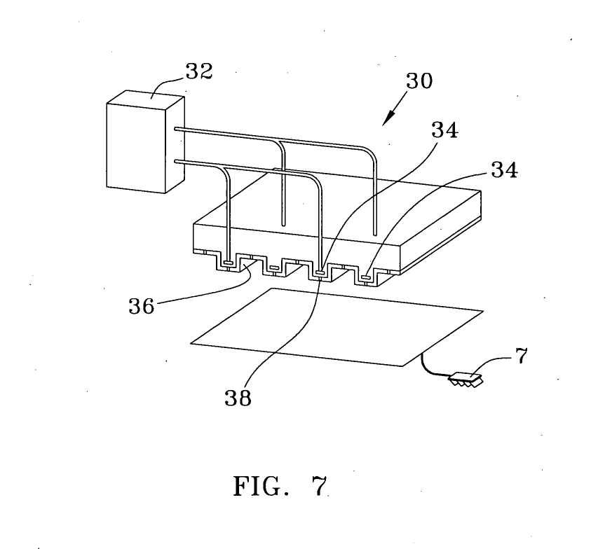

In the preferred embodiment, a miniaturized ionized gas lysis device 30 uses a low temperature, atmospheric pressure system using only a few watts of power. A typical miniaturized planar ionized gas lysis system developed for the creation of chaperone protein-antigen complexes (CPAC) is shown in FIG. 7. The use of flexible dielectric substrates was the major modification in order to achieve an ionized gas-producing design. The new design of the micro-machined Kapton™ membrane eliminated the nonflexible ceramic substrates while maintaining aluminum electrodes. The electrodes are aluminum foil tapes. The flexible ionized gas device 30 can be wrapped around several surface contours.

The preferred embodiment uses a nonequilibrium discharge. The bulk gas temperature increases only slightly, in the realm of a few degrees Centigrade. Essentially, most of the input electrical energy is used in the acceleration of electrons to create the electron avalanche to sustain the corona discharge (plasma or ionized gas). The voltage is low in the preferred embodiment because the corona discharge does not exceed the 1000 micron range.

While there must be approximately a 30 kV/cm electric field applied to sustain a corona discharge in air, the preferred embodiment operates to produce a corona discharge within a very small gap, (i.e. up to 1000 microns) such that the power consumption lies in the range below 2 watts.

The preferred embodiment ionized gas lysing device 30 (FIG. 7) has a low voltage, high frequency, high current electrical power supply 32 that develops an electrical field between a pair of interdigitated electrodes 34 covered in a thin dielectric coating, and that are alternately situated. The spacing between adjacent electrodes 34 is in the realm of 10 microns. With this spacing, the generation of an ionized gas (i.e. plasma discharge) there between is in a generally hemispherical shape. Because of the close proximity between the adjacent interlaced electrodes, the aggregation of all of the hemispherical plasma discharges create a resultant planar plasma discharge (in the range of 1000 microns deep only because of the low voltage used) near which the biological material to be lysed can brought. In this design, the fabrication of a stabilizing corona discharge that suppresses the glow-to-arc transition is accomplished by design of the dielectric coatings 36. These dielectric coatings (of a high dielectric constant material such as found on a polyimide) are of uneven thickness, utilizing perforations 38 (or indentations) in the 10 micron range with a center-to-center distance of 20 microns (shown in FIG. 7). The dielectric coating resides on top of the electrodes that have been deposited on a ceramic substrate. Each of the perforations acts as a separate active current limiting microchannel that prevents the overall current density from increasing above the threshold for the glow-to-arc transition. (The micromachined dielectric layer of can be eliminated if a pulsed discharge is used enabling an electronic pathway to avoid a filamentary discharge. This allows for a stable nonequilibrium “cold” plasma discharge to be produced at atmospheric pressures.) The chosen polyimide for the dielectric coating Kapton™ is micromachine fabricated with a laser-melting instrument. The melting process employed creates funnel-like cuts instead of cylindrical passages.

The preferred embodiment flexible ionized gas device 2 is composed of a dielectric barrier 36 made of two layers of Kapton™ tape with metal (aluminum foil) enveloped electrodes 34 wrapped around the contours of any surface. A cooling system is not needed because the ionized gas is only activated for minutes to effectively ionize gas lyse a biomaterial. This preferred embodiment ionizing gas lysing device 30 uses an atmospheric pressure, room temperature flexible ionized gas device. Direct grounding to the power supply 32 may be utilized, however indirect grounding 40 of the material to be lysed will suffice to achieve the desired results.

Ionized gas reactors can be classified by their physical construction and method of energization. Each physical arrangement has certain advantages for reactions, while the energization is closely coupled with the reactor design. The ionized gas reactor behaves electrically as a capacitor, C, (albeit a 180° flat capacitor) while the secondary windings on the transformer behave as an inductor, L. Therefore, the resonant frequency, F, of the system can be represented by Equation (12):

$$F = \frac{1}{2 π} \sqrt{\frac{1}{LC}} \qquad ( 12 )$$

While the inductance of the system will be fixed with the choice of transformer, the capacitance of the system may vary as objects are placed therein for ionized gas exposure. One can find the capacitance of the reactor by treating the equivalent capacitance (in series) in Equation (13):

$$C = \frac{1}{( 1 / {C}_{d} ) + ( 1 / {C}_{m} ) + ( 1 / {C}_{a} )} \qquad ( 13 )$$

where C_{d} is the capacitance of the dielectric barrier, Cₘ is the capacitance of the material to be decontaminated (either dielectric or metallic), and Cₐ is the capacitance of the air. (It should be noted that the capacitance of the reactor changes tremendously when ionized gas fills the gap. However, we are solving for the onset-corona conditions.)

It can be seen that the main contribution to the capacitance of the system is the dielectric on the electrode, and not the object within the ionized gas region. These results have been verified experimentally. Since the capacitance of the system is related to the applied voltage, V, solving for the capacitive voltage drop, across the dielectric barrier, V₁, can be seen in Equation (14):

$$C = \frac{q}{V} \to {V}_{1} = \frac{V ( {C}_{m} {C}_{a} )}{{C}_{m} {C}_{a} + {C}_{m} {C}_{d} + {C}_{d} {C}_{a}} \qquad ( 14 )$$

As shown in Equation (14), the presence of the dielectric on the electrodes creates a voltage drop for the initiation of the ionized gas. Similarly, the voltage drop reveals that the electric field strength of 30 kilovolts per centimeter of corona onset-potential in air can be achieved more easily with a material having high effective capacitance. Counter intuitively, the placement of various objects within the ionized gas region enhances the corona production process. Further, using a miniaturized system where the electrodes are not placed very far apart implies that the voltages required to achieve 30 kV/cm can be in the order of 3000 volts and smaller while the power consumption can be below 2 watts.

Steps 6 & 7—Second i-DEP Purification and Concentration

In this step (which is only necessary to separate the additionally created CPAC from the unwanted biological materials) the recently lysed solution is again passed through the i-DEP device (as detailed above) and flushed with a flushing solution (generally distilled deionized water). The concentration will passively retain biomaterials such as antibodies and protein complexes including CPAC on different rows of the microstructured array. The novel basis of this approach is that when proteins change their conformation, their dipole moment and polarizability, change slightly, and allow separation due to their migration differences within a nonuniform electric field imposed on the i-DEP microstructured array. Coated electrodes (e.g. 150 nm thick polymer) are placed at the solution influent and effluent and within the micropillars. The micropillars modify the electric field distribution between the two electrodes, creating zones with relatively higher and lower field strengths (nonuniform) due to the applied high frequency power. Different dielectrophoretic responses can be obtained from the same cells depending on the frequency and amplitude of the applied electric. Bioparticle parameters that affect the dielectrophoretic response are geometrical: size, shape, conformation, cell morphology (i.e., presence of a flagellum), surface charge, among others. Additionally the viability (live vs dead) of cells allow i-DEP segregation. The applied voltage on the i-DEP is removed and the purified, concentrated CPAC sample (which larger and much richer in types of CPAC than any of the prior art has demonstrated) is collected.

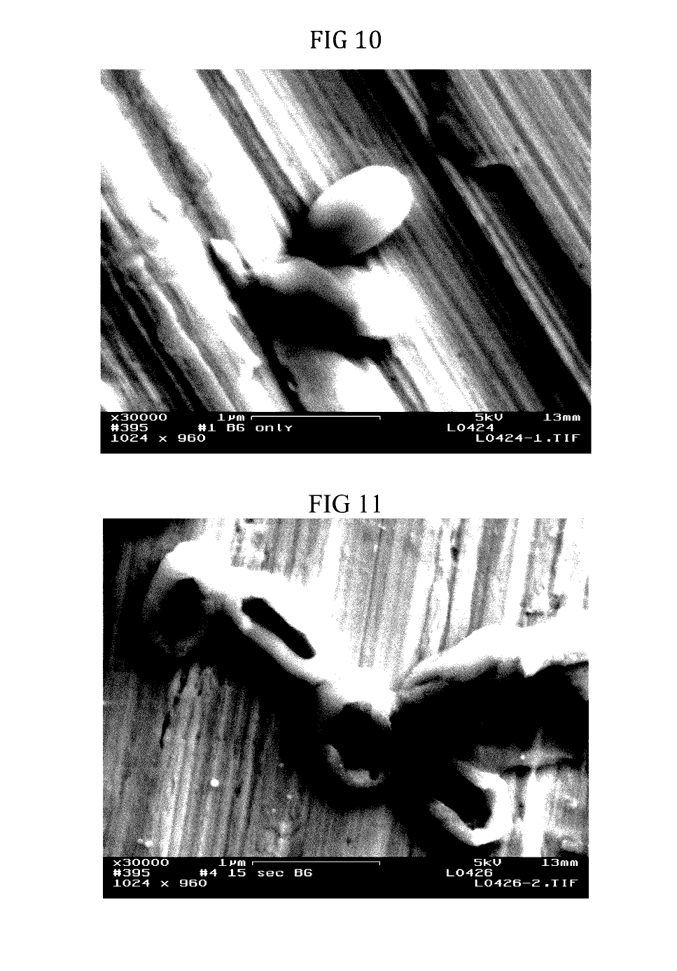

FIGS. 10 and 11 are SEM photographs that show Bg spores prior to ionized gas lysing and after 15 seconds of ionized gas lysing treatment. The cell membrane has been completely breeched allowing access of the CPACs.

Steps 8, 9 & 10—Coating the CPAC with a Biodegradable Polymer for Enhanced Delivery

Nanoparticle-based carriers have demonstrated enhanced, sustained release of antigens at target sites, oriented antigen and/or adjuvant presentation, and specific targeting. The potential for encapsulated and sustained release of antigen within cells has been proposed to increase antigen-presentation by dendritic cells. Sustained release of antigens from particles can induce strong immune protection, eliminating the need for repeated doses of the vaccine (simultaneous priming and boosting). The preferred embodiment encapsulates its vaccine as particles with PGLA to enhance the uptake of antigens and adjuvants by dendritic cells and result in better immune responses compared to its soluble counterparts.

PLGA is a biocompatible and biodegradable material that has been approved as an in vivo substitute to polymeric matrix by the FDA. Antigen encapsulation into PLGA nanoparticles resulted in increased cellular uptake of antigen and induced T cell responses. The mechanism of antigen delivery involved cross-presentation. While micropinocytosis of soluble antigen leads to poor MHC class I presentation by APC, phagocytosis of particle-loaded antigen enhances cross-presentation, leading to potent CTL responses.

The fact that peripheral DCs can capture nanoparticles at peripheral sites, such as epidermis and dermis, it is advantageous to directly target nanoparticles to the lymph nodes (LN). Lymph vessels have diameters around 10-60 microns and the sinusoid in the spleen varies from 150 to 200 nm. Only particle complexes of 20-200 nm can effectively enter in lymphatic system. Particle sizes 20 nm and smaller are taken into dendritic cells at close to 100% efficiency while particles larger than 100 nm are only 10% removed. Particles larger than 200-500 nm do not enter the lymphatic system unless they are associated with dendritic cells. Nanoparticles of less than 200 nm can, therefore, reach the lymphoid organs directly within hours after injection, whereas particles larger than 200-500 nm require dendritic cells, which can squeeze through openings of overlapping endothelial cells and will take approximately 24 hours to arrive in the lymph nodes. The size of the nano particles also seems to influence their cellular uptake mechanism (e.g. endocytosis, micropinocytosis, and phagocytosis) and intracellular pathway. Each endocytic pathway is also defined by a specific size range of engulfed soluble or particulate material. In general, virus-sized particles (20-200 nm) taken up by endocytosis. Larger sized particles are taken up by micropinocytosis and phagocytosis and is restricted to a few specialized cells such as macrophages.

Fabrication of nanoparticles in geometries resembling pathogens ranging from viruses (20-100 nm) to bacteria and even cells in the micrometer range) and the ability to orient pathogen-relevant danger signals on the nanoparticle surface activate APCs and stimulate nanoparticle uptake.

Uptake of nanoparticle loaded antigens by dendritic cells highly depends on physiochemical properties of nanoparticles including size, shape, surface charge, hydrophobicity, and hydrophilicity. These are important parameters that determine biodistribution, cellular interactions, and cellular infiltration. Altered electrostatic or receptor-binding properties facilitate improved interaction with dendritic cells compared to soluble antigens. An optimal particle size for uptake by human blood traveling dendritic cells was under 500 nm. Particle size also has effects on the antigen presenting cells. Particles traffic to the draining lymph node in a size-dependent manner. Large particles (500-2000 nm) are taken up by peripheral antigen presenting cells at the injection site, while small nanoparticles (20-200 nm) are internalized in dendritic cells and macrophages residing in lymph nodes. Smaller nanoparticles can independently diffuse across the interstitium and penetrate the lymphatic system, while delayed transport of larger nanoparticles to lymph nodes supports a requirement for cell transport. Particles of 40-50 nm in size have been shown to elicit stronger T cell responses.

The stability of nanoparticles is also an important factor the rate of antigen release. Polymers such as polylactides (e.g. PLGA) are rapidly hydrolyzed in the body. PLGA particles have a slower antigen release kinetics compared to liposomes. Mice vaccinated with ex vivo stimulated splenocytes from PLGA particles displayed higher interferon (i.e. IFN-γ) secretion compared to splenocytes from liposome. Therefore, kinetics of sustained release from PLGA particles compared to liposomes was thought to account for more effective in vivo CD8+ T cell responses.

The surface charge of nanoparticles profoundly affects the internalization capability. This is due to the negative charge of the cell membrane, which increases the affinity for positively charged materials. Additionally, cationic charged nanoparticles can enhance DC uptake compared to negative charged particles through electronic binding. The inherent adjuvant of nanoparticles is exemplified by cationic liposomes leads to activation of mouse bone marrow dendritic cells.

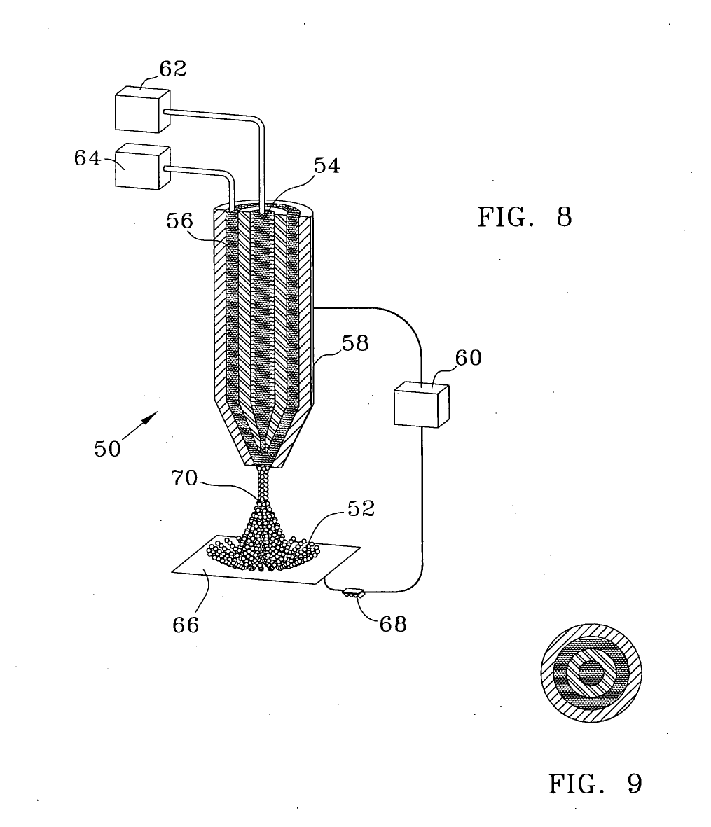

Looking at FIGS. 8 and 9, in the preferred embodiment, the electrospray encapsulation device 50 generates monodispersed serum droplets of CPAC 52 that is encapsulated with a biodegradable coating. The electrospray encapsulation device 50 is a coaxial delivery device having an inner chamber 54 and a concentric, conductive outer chamber 56. The outer housing 58 for the outer chamber 56 is conductive and is in electrical communication with a high voltage, high frequency A/C power supply 60. A first fluid pressure generation means 62, is provided to pump a first fluid (the CPAC laden fluid derived from the ionized gas lysing and extracted from the second i-DEP purification) at a specified flow rate down the inner chamber 54. A second fluid pressure generation means 64, is provided to pump a biodegradable fluid (a biodegradable polymer such as PLGA) down the outer chamber 56 at a specified flow rate. Below, and adjacent the proximal end of the device resides an encapsulated monodispersed droplet collection vessel 66 that is grounded 68 with the power supply 60. The outer chamber 56 has a high frequency high voltage applied thereto.

In operation, a voltage and AC current is selected as mathematically derived through the equations set forth below for the physical specifics of the coaxial syringe used. This voltage and current is applied to the outer housing 58 of outer chamber 56 of a coaxial syringe. The CPAC laden fluid is pumped down the inside of the central inner chamber 54 at a flow rate derived specifically for the coaxial syringe used while the PLGA is pumped down the inside of the outer chamber 56 at a flow rate derived specifically for the coaxial syringe used. These two flow rates will not be the same in most conditions because the volume of the CPAC exceeds the volume of the encapsulation. The encapsulation thickness generally will remain less than 2 nm. Any thicker encapsulation would require a longer period of time for degradation thus reduce the speed of CPAC uptake. As the CPAC fluid reaches the bottom (distal end) of the inner chamber 54, a Taylor cone of fluid 70 is formed. Simultaneously, the PLGA is reaching the bottom of the outer chamber and is forming its own Taylor cone that envelops and forms on the outer surface of the inner, CPAC Taylor cone. The electric charge repulsion on the aggregated Taylor cone causes an encapsulated CPAC droplet 52 to form. The encapsulated monodispersed droplet collection vessel 66 that is grounded 68 with the power supply 60 creates electric field lines of force that draw the charged droplets 52 to the vessel 66. This allows the compilation of the series of electrosprayed extremely small encapsulated droplets (now serum) to be collected in a therapeutic amount. It is to be noted that the high voltage A/C power supply 60 is an adjustable high voltage A/C power supply capable of adjustment of it frequency, waveform, amplitude, current, phase angle, polarity, and pulse width. To achieve the nanoscale size of the serum droplets, the inner chamber's diameter generally will not exceed 100 microns.

The electrospraying of the CPAC in a biodegradable polymer (such as PLGA) is done on a nanoparticle scale that will target both the innate and adaptive immune system. This enables the nanoparticle CPAC to more quickly be taken directly to the lymph node to enhance the immune response. The objective of the electrospraying device and methodology disclosed herein is to create a series of monodispersed drops in the range of 0.001 to 150 microns in diameter.

Typical electrostatic spraying processes are used in the painting and textile industry where large amounts of material composed of droplets with diameters in the 100 micron range with a large distribution of droplet sizes are applied to flat surfaces. The conventional coatings produced are 200 microns thick. The large droplet diameters prevent the evaporation of the solvents in route from the electrosprayer to the substrate. The spray heads for these designs are designed so that the charged material forms large droplets. The liquid jet generators for ink jet printing are a controlled form of electrostatic spraying. In ink jet generators, streams of liquid drops on the order of 75 to 125 microns in diameter are produced and guided by electric fields to the desired location to form the printed character. The prior art does not teach an electrodynamic coater (using high voltage alternating current and voltage with specific waveforms and pulse widths) for applying coatings from 10 angstroms to hundreds of microns uniformly with a large amount of capillary needles at atmospheric pressure.

For a given liquid, stable and monodisperse electrosprays can be established within certain ranges of liquid flow rates and applied voltages for a given cone-jet domain. The droplet size is found to be dependent on the liquid flow rate and the applied voltage. Specifically, the droplet size increases monotonically with the liquid flow rate and decreases with larger applied voltages. As much as 50% variation of the droplet size can be altered at a fixed flow rate by varying the applied voltage. The droplet size is not dependent on the capillary size. However, an increase in the capillary diameter narrows the operating domain for the electrospray. Additionally, this electrospray process is very dependent of the electrical conductivity of the fluid. As the electrical conductivity is increased, then smaller flow rates and particles are generated. Droplet size and monodispersity are independent of the electrode configuration, as long as the system is operated at the onset voltage condition—the minimum condition at which the cone-jet mode is established.

Electrostatic atomization, also known as electrospray, refers to the atomization of a liquid through the Coulombic interaction of charges and the applied electric field. Electrostatic atomization has been of much interest in the scientific community because of its many applications and advantages. Electrostatic atomization offers several advantages over alternative atomization techniques. This is mainly due to the net charge on the surface of the droplets that is generated and the Coulombic repulsion of the droplets. This net charge causes the droplets to self-disperse, preventing their coalescence. The trajectory of a charged droplet can be guided by an electrostatic field. The advantage of this type of atomization is the ability to control the size distribution of the spray and under specific operating conditions, obtain a monodisperse spray. Because of these advantages, there are a wide number of applications where electrostatic atomization can be used. A few of these applications include spray painting, drug inhalation therapy, and ink jet printing.

Electrospray can be described by three different processes. The first process is the formation of the liquid meniscus at a capillary tip which results from a number of forces acting on the interface, including surface tension, gravitational, electrostatics, inertial, and viscous forces. Sir Geoffrey Taylor was the first to calculate analytically a conical shape for the meniscus through the balance of surface tension and electrical normal stress forces which we now know is called the “Taylor cone” in electrospray and appears in the cone jet mode.

The cone jet mode is one of the most widely studied and used modes of electrospray. In the cone-jet mode liquid leaves the capillary in the form of an axi-symmetric cone with a thin jet emitted from its apex. The small jet of liquid issuing out of the nozzle is electrostatically charged when subjected to an intense electric field at the tip of the capillary nozzle. In this case, the droplets are approximately 10 microns in diameter and difficult to visualize with standard macro photography. The charged droplets are propelled away from the nozzle by the Coulomb force and are dispersed out as a result of charge on the droplets.

The key to achieving the electrospray coating of the CPAC within the parameters outlined is based on the injection of the droplets and control of the atomization process with an electric field. This specific approach uses ion liquid propulsion for injection of the droplets from the injection ports and dielectrophoretic (DEP) force acting on the liquid-gas interface to control the atomization and prevention of satellite droplets. Both ion liquid propulsion and DEP control of the interface are accomplished using the same set of microinjectors and electrodes.

Ion drag pumping is not new and neither is the use of DEP force for manipulation of flow field and particle or droplet trajectories within the flow. The electric field induced pressure gradients for driving the flow can be written as seen in equation 15:

$${f}_{E} = Q \vec{E} - \frac{1}{2} {E}^{2} ∇ ɛ - ∇ [ \frac{1}{2} ρ {{E}^{2} ( \frac{\partial ɛ}{\partial ρ} )}_{T} ] \qquad ( 15 )$$

where ∈ is the dielectric permittivity of the fluid, ρ is the mass density, Q is the electric field space charge density, T is the temperature, and E is the applied electric field strength. The first term in the right hand side of equation (2) represents the force on the free charges present and gives rise to the so called Coulomb force, which is the primary driving force in most ion-drag pumps for pumping a liquid or gas in the single-phase mode. The second and third terms are the electrostrictive force and the dielectrophoretic (DEP) force and will be discussed in the following paragraph in more detail. At this point, we are not too concerned about the electrostrictive force and we will not be exploiting this force in this innovation. In this equation, Q is the space charge density and is defined as follows in equation 16:

$$Q = \frac{I}{( u + μ E ) A} \qquad ( 16 )$$

where, I is the current, u is the bulk fluid velocity, μ is the ion mobility, E is the field strength, and A is the flow cross-sectional area. In the simplest form for a laminar flow within a circular flow cross-section, one may derive a relationship between the pressure rise produced by the pump, the driving voltage and the pump geometrical parameters for the bulk velocity dependence on the current and voltage as well as the relationship between voltage and current (see equation 17):

$$Δ p = \frac{ɛ {u}^{2}}{6 {μ}^{2}} \qquad ( 17 )$$

where u is average droplet escape velocity in m/s, μ is the ion mobility in m²/Volt-sec, and P is the permittivity in C/volt-m. It turns out that to solve for velocity in terms of the applied electric field strength, the pressure gradient becomes almost similar to the famous equation for electrostatic precipitators.

DEP force exists when the following two conditions are simultaneously satisfied: (a) there is a gradient of the electric field strength, and (b) there is a change in the dielectric constant across the interface separating the two phases. The DEP force experienced by a droplet is calculated from the following equation 18:

$${F}_{e} = \frac{π}{4} {d}^{3} {ɛ}_{o} {k}_{1} [ \frac{{k}_{2} - {k}_{1}}{{k}_{2} + 2 {k}_{1}} ] \cdot ∇ {\vert E \vert}^{2} \qquad ( 18 )$$

where d is particle diameter, ∈₀ is dielectric constant in vacuum, k is the relative dielectric constant, and E is the electric field strength. K is 1 for air and approximately 2 for most other heavy liquid fuels. Therefore, there is a significant change in the dielectric constant giving rise to measurable force acting on the interfaces between these two fluids (i.e., air and liquid fuel). It is clear that if any external force is to be effectively used for formation of droplets, it should act near and on the liquid-air interface, at the point where the droplets are being formed. This process would prevent excessive pressure loss in the system as well as provides more local control on formation of droplets. The results indicate that using an E-field force for controlling atomization and suppression of satellite droplets by directly interacting with the liquid-gas interface converge.

A droplet size of 10 nm without daughter satellites has been produced. The surprising result was that the selection of specific electrical conditions such as frequency, waveform, and intensity produced different droplet distributions at will.

Electrospray has been shown to generate a very narrow size distribution of droplets. Based on the theory of electrohydrodynamics (EHD), the key to electrostatic spraying is the electric stress on the liquid interface. A strong enough electric field can produce instabilities in the interface resulting in breakup of the liquid jet issued out of the nozzle and emission of fine droplets from the jet. The cone-jet mode can experience both kink and varicose instabilities very similar to a natural jet break-up or Rayleigh instability, depending on the ratio of the electric normal stress over the surface tension stress. As this ratio increases with increasing flow rate and electric stresses, the number of satellite droplets formed increases. In general, a bimodal distribution of the droplets is shown to exist while the satellite droplets are forced to the periphery of the spray.

For a given amount of liquid accumulated at the tip of a capillary needle, the minimum voltage or threshold voltage corresponding to the breakup of the liquid meniscus was determined. For example, for a droplet of 0.4 μl, 4500 VDC is needed for atomization. For a given meniscus volume at the tip of the atomizer, the maximum voltage pulse amplitude has to exceed a critical amount for atomization to take place and for a given voltage, the drop volume cannot exceed a maximum volume for atomization to take place.

This phenomenon is easily explained through a force balance, where the surface tension force is the stabilizing force and the electric stresses are the destabilizing part of the equation. As the volume of the droplet decreases, the surface tension acting on the liquid meniscus increases. Because of this increased surface tension, the electric field intensity acting on the drop has to be increased in order for atomization to occur. This approach to electrostatic atomization is very unique and the foundation of the current proposal. Our results and analysis show that by pulsing the electric field, spray-on-demand is possible.

The electrospray capillary orifice or bore is typically two orders of magnitudes larger than the cone jet and the monodispersed droplet diameter. In order to establish the monodispersed submicron droplet generation, (glass, Teflon, or other dielectrics) capillaries are coated with a conductor. The tip of the capillary may be flat or an angle to produce the droplets. The capillary bore combined with the flow rate of fluid (head pressure) at the selected electrical conditions produces the desired droplet monodispersity and diameter.

Most of the atomization modeling, is based on empirical correlations and results obtained by other investigators for electrospray. The critical parameters for design are the operating parameters that allow the minimum amount of power for the given flow rates. For electrospray atomization to take place, the electrical relaxation time of the fluid has to be much shorter than the hydrodynamic transit time of the fluid flow. That is, the following inequality has to be satisfied (see equation 18):

(tₑ=β∈ₒ/K)<<(tₕ−L/U) (19)

where tₑ is the electrical relaxation time of the fluid and tₕ is the hydrodynamic time of the fluid flow, β, ∈ₒ and K are the relative permittivity, permittivity in vacuum, and electrical conductivity of the fluid, respectively, L is the axial characteristic length scale, and U is the characteristic jet velocity. Using this relationship, one is able to find the most appropriate combination of fluid property (conductivity), flow rate, and flow geometry. In fact, one of the outcomes of this relationship is that for a given fluid and nozzle, there is an upper limit to the flow rate of the liquid to ensure electrospray atomization. However, Equation 19 reveals that as the flow rate increases, the higher should the conductivity of the liquid be. For very low conductivity dielectrics such as hydrocarbon fuels, one can add trace amount of an additive such as Stadis 450 (DuPont) to increase the electrical conductivity without significant change in other properties. Further, the right choice of electrodes for spray may relax the criterion to the point that even if the above criterion has not been met, electrospray can be obtained at high flow rates. When using polar liquids with large dielectric constants (such as alcohol) or high viscosity non-polar liquids (such as saline solution), the current varies as shown below in equation 20:

$$I / {I}_{o} ≅ {6.2 [ \frac{Q}{{( β - 1 )}^{1 / 2} {Q}_{o}} ]}^{1 / 2} - 2.0 \qquad ( 20 )$$

where I is the current (in A), Q is the flow rate out of the nozzle (m³/s), and β is the surface tension of the fluid (in N/m). Iₒ is the dimensionless current and Qₒ is the dimensionless flow rate as defined below in equation 21:

$${I}_{o} = {( \frac{{ɛ}_{o} {γ}^{2}}{ρ} )}^{1 / 2} \\ {Q}_{o} = {γɛ}_{o} / ρ K . \qquad ( 21 )$$

The maximum charge on droplets, qₘₐₓ, is set by the well-known Rayleigh Limit (RL) as described below in equation 22:

q̅ₘₐₓ=π(8∈ₒγd³)^{1/2} (22)

The limit of the current leaving in form of charge on the droplets is then the product of the RL and the number density of droplets leaving the jet per unit time (flux) in equation 23:

$${I}_{max} = 12 \sqrt{2} {( \frac{{ɛγ}_{o}}{{d}^{3}} )}^{1 / 2} Q . \qquad ( 22 )$$

The above calculations are integrated into fluid dynamics modeling to predict the sizes and percentages of the droplets to be produced and the power requirements.

If this equation is combined with equations (22 and 23) a relationship between the flow rate and droplet size can be developed in equation 24.

d=[0.365(K/∈ₒ)^{1/2}(β−1)^{-1/4}Q^{-1/2}−0.118(γ/ρ)^{1/2}Q⁻¹]^{-2/3} (24)

The atomizer produces extremely small droplets (<10 micron) using very low driving pressure. While conventional atomizers required a significant pressure to atomize the liquid, the electrospray or e-field atomizer does not. It turns out the power requirements for electrospraying are less than 0.5 W for 1 ml/s of fluid sprayed.

In an ideal method and apparatus of preparation of nanoparticles one should be able: 1) to control the average size of the nanoparticles; 2) to obtain a very narrow distribution of sizes; 3) to passivate the surface and eliminate surface states; and 4) to control the shape of the particles. One of the possible ways to solve these problems simultaneously is to restrict the reaction volume in which the particles are created.

### Step 11—Host Reintroduction Formulation Administration and Dosage

The immune system protects a host against pathogens by mounting an immune response which is specific to an antigen of an invading pathogen. The objective of immunization is to elicit an early protective immune response by administering to the host an antigen associated with a pathogen.

Timing is everything. One of the major benefits of the personal serum developed by the apparatus and methods disclosed herein is that it may be reintroduced into the host at a much later date than conventional serums and still meet with success.

One major challenge in developing effective vaccines is to design a vaccine that can induce an effective immune response to the desired antigen with no or limited side effects. The ability of chaperone proteins such as heat stress proteins (hsp) to escort antigenic peptides that interact with antigen presenting cells (APC) through a receptor and stimulate APCs to secrete inflammatory cytokines while mediating the maturation of dendritic cells. These chaperone proteins (e.g. hsp) enable the utilization of chaperone proteins-antigen complexes to develop a new generation of prophylactic and therapeutic vaccines against cancers and infectious diseases. These personalized vaccines promise limited side effects.

The resultant pharmaceutical composition that is produced by the disclosed apparatus and methods, is comprised of an effective amount of a chaperone proteins, chaperone protein complexes, CPAC, or aggregates thereof. The composition can be administered with a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. These compositions can take the form of solutions, suspensions, emulsion, tablets, pills, capsules, powders, sustained-release formulations and the like. These compositions can also be formulated as a suppository.

Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

The amount of the pharmaceutical composition which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances.

Dosages of chaperone protein complexes can be in the range of 1-1,000 micrograms. Preferably the dosage range is 25-500 micrograms. Most preferably the dose range is 1-50 micrograms. A single dose of vaccine may be used, or an initial vaccination may be followed by additional boosters. Moreover, therapeutic regimens and pharmaceutical compositions can be used with additional immune response enhancers or biological response modifiers including, but not limited to, interferon (IFN)-α, IFN-β, IFN-γ, interleukin (IL)-1, IL-2, IL-4, IL-5, IL-6, IL-12, IL-15, granulocyte-macrophage colony-stimulating factor (GM-CSF) or tumor necrosis factor (TNF)-α. The immune response enhancers or biological response modifiers can be provided as proteins or nucleic acids encoding the proteins for the appropriate immune response enhancer or biological response modifiers in combination with the chaperone protein complexes recovered by the methods and apparatus of the invention. Formulations for administration via a route such as, but not limited to oral, parenteral, intravenous, intraperitoneal, mucosal, or intradermal, for inhalation, nasal drops, topical gels, and slow release formulations, and preferred dosages thereof are provided for the treatment and prevention of cancer, such as primary and metastatic neoplastic diseases, the treatment and prevention of infectious diseases, and adoptive immunotherapy.

The vaccines of the present invention may be designed for administration to any mammal including, but not limited to, humans, domestic animals, such as cats and dogs; wild animals, including foxes and raccoons; livestock and fowl, including horses, cattle, sheep, turkeys and chickens.

Those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

1. A vaccine for an intended vaccine recipient comprising:
a collection of chaperone protein-antigen complexes selected from the group consisting of hsp 27, hsp28, (s-hsp), hsp40, hsp60, hsp70, hsp72, hsp 84/hsp86, hsp90, hsp100, hsp110, defensin, calreticulin, cathelicidins, BiP/grp78, grp75/mt, gp96, tumor suppressor P53, p21 CDK inhibitor, extracted foreign DNA, and other peptides and proteins formed by exposure to an ionized gas including reaction byproducts such as peroxides, nitrogen oxides, and reactive oxygen species;
wherein said vaccine is a personal targeted immunotherapy vaccine where said chaperone protein antigen complexes are derived from infected cells taken from said intended vaccine recipient.

2. The medical vaccine of claim 1 wherein said chaperone protein-antigen complexes derived from said intended vaccine recipient are derived through ionized gas lysing of said infected cells and other tissue-derived lysates.

3. The medical vaccine of claim 1 wherein said vaccine is coated with a biodegradable polymer coating enveloping at least one molecule of said chaperone protein-antigen complexes, and wherein said thickness of said biodegradable polymer enveloping said chaperone protein-antigen complex is between 0.5 nanometer and 1000 nanometers.

4. The medical vaccine of claim 2 wherein said chaperone protein-antigen complexes derived through ionized gas lysing is purified by an insulator-dielectrophoreisis device

5. The medical vaccine of claim 3 wherein said chaperone protein-antigen complexes have a mean diameter of 0.1 to 5 nanometers.

6. A method for generating a targeted immunotherapy vaccine of chaperone protein-antigen complexes that are used, upon reintroduction into a donor's body, to generate T cells and other cells reactive to a chaperone protein-antigenic complex molecules, said method comprising the steps of:
extracting biological material from an intended vaccine recipient;
passing said biological material through an insulator-dielectrophorectic device's microstructured array to separate and concentrate biological materials including migrating and tumor cancer cells and pathogens;
extracting said concentrated biological materials;
in vitro, ionized-gas lysing of said concentrated biological materials to produce a chaperone protein-antigenic complex by the noncovalent interaction of an antigen molecule and a chaperone protein; and
extracting said chaperone protein antigenic complexes.

7. The method for generating a targeted immunotherapy vaccine of chaperone protein-antigen complexes of claim 6 further comprising the step of:
passing said extracted chaperone protein-antigen complexes through said insulator dielectrophorectic device's microstructured array to separate and concentrate said extracted chaperone protein-antigen complexes; and
extracting said concentrated chaperone protein antigen complexes.

8. The method for generating a targeted immunotherapy vaccine of chaperone protein-antigen complexes of claim 7 further comprising the steps of:
electrospraying a coating of a biodegradable polymer on said concentrated chaperone protein-antigen complexes; and
collecting said coated, concentrated chaperone protein-antigen complexes.

9. The method for generating a targeted immunotherapy vaccine of chaperone protein-antigen complexes of claim 6 further comprising the steps of:
electrospraying a coating of a biodegradable polymer on said chaperone protein antigen complexes; and
collecting said coated chaperone protein antigen complexes.

10. A method for generating a targeted immunotherapy vaccine of chaperone protein-antigen complexes that are used, upon reintroduction into a donor's body, to generate T cells and other cells reactive to a chaperone protein-antigenic complex molecules, said method comprising the steps of:
extracting biological material from an intended vaccine recipient;
ionized-gas lysing of said biological materials to produce a chaperone protein-antigenic complex by the noncovalent interaction of an antigen molecule and a chaperone protein; and
extracting said chaperone protein-antigenic complexes.

11. The method for generating a targeted immunotherapy vaccine of claim 10 further comprising the steps of:
electrospraying a coating of a biodegradable polymer on said chaperone protein-antigen complexes; and
collecting said coated chaperone protein-antigen complexes.

12. The vaccine of claim 2 wherein the chaperone protein complexes are present in aggregates that have a molecular weight that is greater than 300 kDa.

13. The method for generating a targeted immunotherapy vaccine of claim 2 wherein said nano particle sized drops have a mean diameter less than 150 microns.
